(19) United States
(12) Patent Application Publication
(10) Pub. No.: US 2015/0218587 A1
(43) Pub. Date: Aug. 6, 2015

(54) Genetic Material Manipulation and Cell Line Creation Techniques and Products Thereof

(71) Applicant: Geneforge Limited, Hong Kong (HK)

(72) Inventor: Yao-Xiong HUANG, GuangZhou (CN)

(73) Assignee: GENEFORGE LIMITED, Hong Kong (HK)

(21) Appl. No.: 14/536,943

(22) Filed: Nov. 10, 2014

Related U.S. Application Data
(63) Continuation-in-part of application No. 13/684,490, filed on Nov. 23, 2012, now Pat. No. 8,883,505.
Application No. 13/684,490 is a continuation-in-part of application No. 12/622,427, filed on Nov. 19, 2009, now Pat. No. 8,318,494.

(30) Foreign Application Priority Data
Nov. 19, 2008 (CN) 200810219234X
Nov. 17, 2009 (CN) PCT/CN2009/074998

(51) Int. Cl. C12N 15/87

(52) U.S. Cl. CPC C12N 15/87

(57) ABSTRACT

The presently claimed invention applies to a genetic material processing, manipulation and transfection method and related product. The claimed invention relates to a method for changing the inherited characteristics of a cell through micro-beam chromosome modification. In one preferred embodiment, improvements to ‘genomic surgery’ are applied to modify source cell genetic material (101). Upon micro-beam welding of combined genetic material (301), transfection takes place either by using a laser beam with a wavelength of 337 nm to perforate a hole (303) on receptor cell (305) or micro-tube control technology is then applied to carry combined genetic material (301) through the micro-pore (303) into the receptor cell (305), inserting the welded chromosome segment (301) by injection. Combined genetic material may be placed anywhere within receptor cell (305), including transfection through nuclear hole (307) into the cell nucleus (311). The presently claimed invention provides a high quality alternate approach to directed genetic recombination without requiring the use of restriction enzymes and is used for chromosomal repair, removal of defects and new organism creation.

### CROSS REFERENCE TO RELATED APPLICATIONS

This application is a continuation-in-part of and claims priority to co-pending U.S. patent application Ser. No. 13/684,490 filed on Nov. 23, 2012 which claims priority to U.S. Pat. No. 8,318,494 filed on Nov. 19, 2009 entitled “Genetic material and chromosomal processing and manipulation methods” the disclosures of which are hereby incorporated by reference, which claims priority to China Application Number 200810219234X filed on Nov. 19, 2008 and to PCT application No. PCT/CN2009/074998 filed Nov. 17, 2009.

### BACKGROUND OF INVENTION

1. Technical Field

The claimed invention is related to genetic technology, particularly involving the creation of new cell lines and processes for genetic material and chromosomal modification and manipulation.

2. Description of Related Art

In terms of artificial chromosome creation, currently there are yeast artificial chromosome (YAC), bacterial artificial chromosome (BAC), plant artificial chromosome (PAC), mammalian artificial chromosome (MAC) and human artificial chromosome (HAC). Known methods for preparation of artificial chromosomes use in vitro recombination techniques with isolated DNA, synthesizing genetic regions by linking together individual base pairs, building base by base to create an entire synthetic chromosome. Current approaches require high time and labor, resulting in extremely expensive efforts to create even a single chromosome.

Known methods exist for DNA manipulation, but the current methods have inherent limitations. Non-specific irradiation techniques such as those disclosed by Vorobjev et al are distinguishable due to lack of precision and high mutagenic effect. These techniques have a number of challenges including difficulties to control and access target chromosomes, as well as the inability to repair or remove defective chromosomes. Current chromosomal transformation methods generally use chemical enzyme digestion to acquire chromosome fragments followed by assembled by enzymes or non-specific action by radiation like Vorobjev. These methods require the preparation of specific enzymes over many procedures to manipulate many chromosome fragments. Moreover, due to difficulty in manipulation of and insertion near telomeres and other difficult to access insertion points, the success rate is not high at present using existing techniques. As a consequence, alternate approaches to genetic material micromanipulation are desirable.

When screening for gene function, current techniques suffer from major limitations. Using the “shotgun approach” has the disadvantage of considerable blindness. When one gene is directly isolated from donor cell DNA, i.e., with a restriction enzyme donor cell DNA is separated into a plurality of DNA fragments, and then transfected into recipient cells by various carriers, so that the DNA fragments of the donor cells are amplified in a large number of copies, substantial time and effort is required to identify the desired target from the hundreds of thousands of DNA fragments in the DNA isolated with the target gene.

In addition to current limitations in artificial chromosome creation, known methods for genetic material transfection share substantial limitations, including random genetic reincorporation, loss of genetic material and lack of precision and refinement in genetic material transfection. Techniques known in the art such as electroporation involve broad and traumatic cell wide disruption of the cellular membrane, leaving successful transfection to chance rather than controlled and directed user guidance.

In addition, current artificial chromosome transfer methods also have problems. A transfer method such as liposome-mediated cell fusion is liable to cause damage to the artificial chromosome. Micro-cell mediated chromosome transfer using large artificial chromosomes relies on random integration of a host cell and as a result the efficiency of the transfer is very low.

### BRIEF SUMMARY OF THE INVENTION

According to the presently claimed invention, improvements to novel methods of chromosomal manipulation, modification and transfection are hereby disclosed. By applying micro-beam techniques, chromosomes are cut and manipulated to a very fine degree of control. Chromosomal transfection is also further improved through guided cellular transfection techniques using directed reinsertion through micro-beam manipulation or micro-injection. Additional improvements of genetic material manipulation and composition include the use of CRISPR/CAS9 for genetic material cutting as well as micro-beam guided genetic material inversions.

The presently claimed technique and related product has several advantages over existing methods, including improved screening for desired genetic characteristics. Since any complex physiological phenomenon is often the result of interaction of multiple genes, a single gene may act only on certain aspects of biological phenomena. Unlike the high volume, low throughput ‘shotgun approach’, the claimed chromosomal combination has the benefit of being a natural gene carrier, resulting in a biological change which is more complete than random single gene insertion. In addition, the claimed technique is ‘lossless’ with no mechanical contact and allows for specific orientation of key genetic components including telomeres, centromeres and replication origin sites in any desired position. Not only is the success rate of transfection improved, but exogenous gene expression can be enhanced as well due to insertion of full-length upstream and downstream genetic control regions and introns. Unlike present synthetic methods, very large genetic constructs are assembled with significantly less cost and time than current synthetic methods building up from the base pair.

Through the use of the presently claimed invention, genetic material is modified so that living cells are modified to alter life activities and functions by control of cellular metabolic processes and alter gene transcription. Moreover, in particular embodiments, desired chromosomes, chromosome fragments, or modified genetic material of exogenous origin are introduced into cells so that new genes are expressed and with cell division the newly introduced traits are passed to progeny cells.

### BRIEF DESCRIPTION OF THE SEVERAL VIEWS OF THE DRAWING

FIG. 1 is the application of the presently claimed invention by microscopic view of the process as applied to a target chromosome.

FIG. 2 (a) shows source genetic material with arrow indicating cutting site for cutting step.

FIG. 2 (b) shows transporting source genetic material to a target location adjacent to target genetic material.

FIG. 2 (c) shows welding of source genetic material to target genetic material.

FIG. 2 (d) shows combined genetic material after welding with the welding point indicated by arrows.

FIG. 3 is an illustration of transfection according to the claimed invention.

FIG. 4 is a chromosome manipulation schematic diagram.

FIG. 5 is a flow diagram according to the claimed invention.

FIG. 6 is a flow diagram according to the claimed invention.

### DETAILED DESCRIPTION OF A PREFERRED EMBODIMENT

The following examples and drawings depict an implementation of the presently claimed invention in further detail. In a first illustrative example:

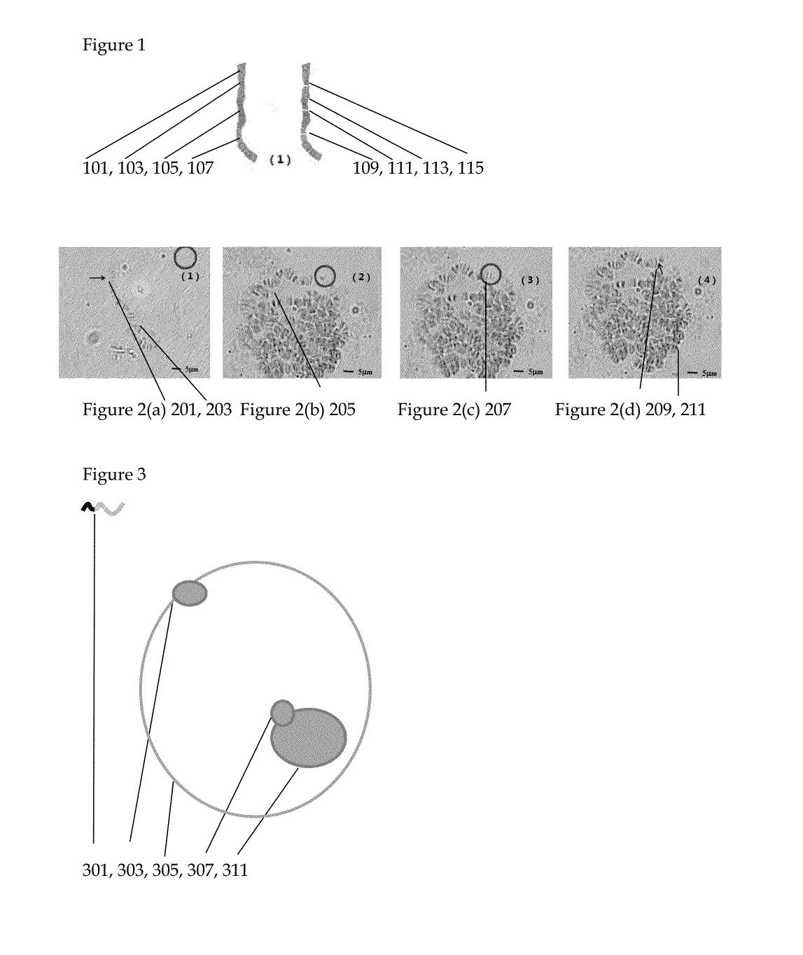

FIG. 1 is an illustrative example of the claimed cutting step. Source genetic material (101) is isolated and desired cutting locations (103, 105, 107) are isolated. In the illustrative example of FIG. 1, cutting is performed at cutting sites (109, 111, 115). In a very simple example of a genetic inversion, isolated genetic material (113) is inverted (turned 180 degrees) and reattached in a subsequent genetic material micro-beam welding step. In an alternate illustrative example, exogenous (or source) genetic material can be inserted at any desired cutting site such as cutting site (109). The genetic material in FIG. 1 is for illustration purposes only as the technique can be performed on any type of genetic material, including mammalian, plant, insect and synthetic. The cutting performed in FIG. 1 is illustrative and can be achieved by both micro-beam cutting as well as use of CAS9/CRISPR endonucleases.

FIG. 2(a)-(d) illustrates genetic material cutting and moving to create combined genetic material on Drosophila chromosome. FIG. 2(a) arrow identifies cutting site (201) and source genetic material (203). FIG. 2(b) depicts transporting source genetic material to target genetic material (205). FIG. 2(c) depicts genetic material welding location (207) at the circle. FIG. 2(d) shows welding point (209) on the combined genetic material (211). The chromosomes in FIGS. 2(a)-2(d) are for illustration purposes only as the technique can be performed on any type of genetic material.

Cutting step is illustrated by FIG. 2(a) which shows source genetic material (203) containing chromosomes prepared for cutting. Source genetic material (203) is placed in an inverted microscope stage (not shown). Using a wavelength of 337 nm, the pulsed nitrogen laser beam is focused through the microscope objective to a micro-beam with a diameter of 0.6-3 microns in diameter. The energy density of the beam is adjusted to 168×10⁶ J/m². Values provided for energy density, power density and micro-beam diameter are for illustrative purposes only and not by way of limitation, as energy, power and beam width can and will vary due to variability in genetic material size and properties.

Transporting step and positioning and alignment of the cut fragment for contact with the second chromosome is illustrated by FIG. 2(b) where laser irradiation used for cutting is turned off and another laser with wavelength of 1064 nm from a continuous single-mode Nd: YAG laser is introduced into the microscope (not shown). The Nd: YAG laser is focused to a micro-beam with a diameter of 0.6-3 microns by the objective of the microscope, so its power density is 63×10 ⁹W/m² when its output power is adjusted to 50 mW and acts as optical tweezers. Through the use of the optical tweezers the cut down chromosome segment is captured, and then moved close into position for welding to target genetic material (205).

Welding step is illustrated by FIG. 2(c) where a wavelength of 337 nm pulsed nitrogen laser beam (not shown) is focused through a microscope objective (not shown) into a micro-beam with a diameter of 0.6-3 microns in diameter. In the illustrative example, the beam's energy density is adjusted to 152×10⁶ μm² but beam diameter and energy properties will vary as physical parameters of the genetic targets change. In the illustrative example, the two chromosomes are radiated with the light beam for 18 seconds, after which the two chromosomes are firmly welded at welding location (207) to one to create combined genetic material. FIG. 2(d) shows welding point (209) on the combined genetic material (211). Welding time will vary due to physical properties of the targets.

FIG. 3 is an illustration of transfection according to the claimed invention. Transfection step uses a laser beam with a wavelength of 337 nm and an energy density of 210×10⁶ μm² to perforate a hole (303) on receptor cell (305). In one embodiment laser tweezers are used to transfect combined genetic material (301) through the hole. In an alternate embodiment, micro-tube control technology is then applied to carry combined genetic material (301) through the micro-pore (303) into the receptor cell (305) and injects the welded chromosome segment by injection. Combined genetic material may be placed anywhere within receptor cell (305), including transfection through nuclear hole (307) into the cell nucleus (311).

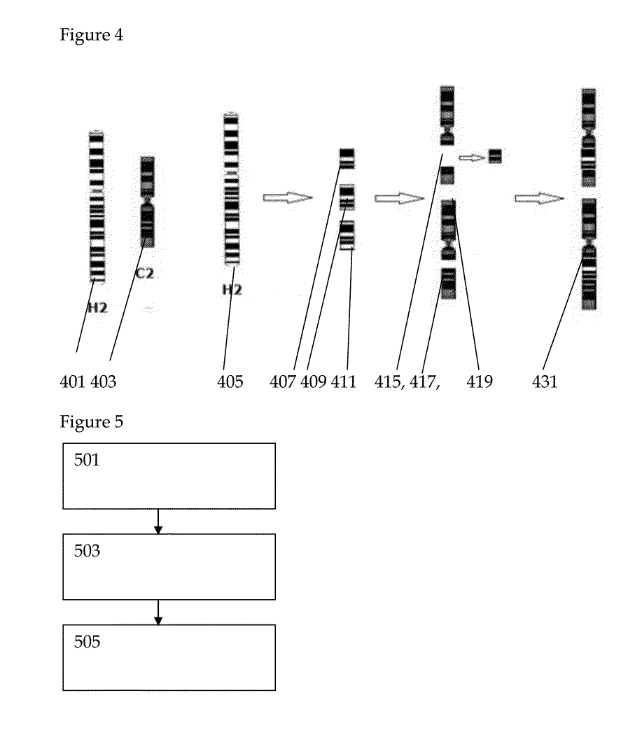

FIG. 4 illustrates a schematic diagram of the human chromosome #2, H2 (401) and CHO cell chromosome 2, C2 (403) cut by laser cutting into different lengths of fragments (407, 409, 411) in H2 (405) with 2q22-2q24 to form an artificial CHO/Human hybrid (431) chromosome. In this illustrated example, Human chromosome number 2 (405) is the second largest chromosome, with 8% DNA and the object of genomic sequence analysis and attention for gene therapy due to the occurrence of AIDS, Alzheimer's and other dementia-related ailments. H2 (405) length is longer and has clear differences between the length of the arm, is easier to identify and manipulate into identifiable fragments (407, 409, 411) without staining under a phase-contrast microscope objective. H2 segment 2q22-2q24 contains tumor necrosis factor, lymphocyte antigens, enzymes and genes associated signal path, and residing in the middle of the long arm of H2, is relatively easy to cut and manipulate.

By applying the claimed technique, laser cutting technology is used to cut next to 2q22-2q24 segment of chromosome to create fragments of different lengths (407, 409, 411).

By applying laser tweezers and laser welding technology, the desired fragment is replaced or embedded assembled into CHO cells (Chinese hamster ovary fibroblasts) on the long arm of chromosome 2, by cutting at desired locations (415, 417, 419). After preparation of artificial chromosome (431), it is transferred into the CHO cell nucleus (not shown) by lossless host cell transfer technology using optical tweezers, laser scissors and light microscopic manipulation and/or micro-injection. Different cutting lengths of the long arm of Human chromosome 2 and the selection of CHO cell chromosome for preparing artificial chromosomes is by way of illustration only and not by limitation. The claimed technique and related product provide for the preparation and transfer of new artificial chromosomes and is a simple, efficient and reliable means for biophysical transformation, equally applicable on a wide variety of cell lines, genetic materials and target transfection hosts.

FIG. 5 is a flow diagram according to the claimed invention. Target cell (not shown) is transfected by cellular micro-pore creation step (501), nuclear pore creation step (503) followed by genetic material insertion step (505) which transfects the genetic composition (not shown) into the target cell (not shown). Micro pore creation (501, 503) and insertion step (505) may be performed by using micro-beam energy or alternately by way of micro-injection.

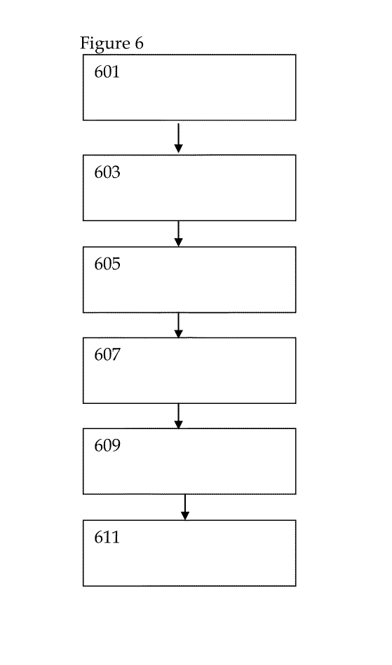

FIG. 6 is a flow diagram according to the claimed invention. Genetic material securing step (601) is followed by cutting step (603) which cuts source genetic material (not shown). Source genetic material is then acquired during genetic material acquisition step (605). Transporting step (607) positions the genetic material fragments (not shown) for joining during welding step (609). After welding, the newly joined genetic material composition can optionally be inserted into a target cell (not shown) by transfection step (611).

The illustrated examples depict selected ways to implement the presently claimed invention, but the presently claimed invention may also be applied in a manner not covered by the above-mentioned cases. The examples are provided by way of illustration and not by restriction of the implementation of the claimed invention. Other approaches may also be applied which do not deviate from the essence and spirit of the presently claimed invention. Foreseeable changes, modifications, substitutions, combinations or simplifications can be applied as equivalent methods and are included in the presently claimed invention within the scope of protection.

I claim:

1. A genetic recombination method, comprising the steps of:
securing source genetic material,
cutting source genetic material,
acquiring cut source genetic material,
transporting cut source genetic material to a target location adjacent to target genetic material, and
micro-beam welding said source genetic material to said target genetic material to create combined genetic material.

2. The combined genetic material product created by the process of claim 1.

3. The method of claim 1 additionally comprising a transfecting step after said micro-beam welding step transfecting said combined genetic material into a target cell.

4. The method of claim 3 wherein said transfecting step uses micro-beam energy to insert combined genetic material into said target cell.

5. The method of claim 3 wherein said transfecting step uses microinjection to insert said combined genetic material into said target cell.

6. The target cell product created by the process of claim 3.

7. The method of claim 3 wherein said source genetic material is exogenous to said target genetic material.

8. The method of claim 1 additionally comprising a transfecting step after said micro-beam welding step transfecting said combined genetic material is inserted into an artificial cellular structure.

9. The method of claim 1 additionally comprising a translocation step after said micro-beam welding step inserting said combined genetic material into a non-cellular substrate.

10. The method of claim 1 wherein said cutting source genetic material is performed by one or more CAS9/CRISPR endonucleases.

11. The method of claim 1 additionally comprising a genetic material inversion step after said cutting step wherein source genetic material is inverted prior to said micro-beam welding.

12. The method of claim 1 wherein said source genetic material is synthetic genetic material.

13. The method of claim 1 wherein said target genetic material is synthetic genetic material.

14. The method of claim 1 wherein said source genetic material and target genetic material is synthetic genetic material.

15. A genetic recombination method, comprising the steps of:
securing source genetic material,
cutting source genetic material,
acquiring cut source genetic material,
transporting cut source genetic material to a target location adjacent to target genetic material,
micro-beam welding said source genetic material to said target genetic material to create combined genetic material and
transfecting said combined genetic material into a target cell nucleus.

16. The combined genetic material product created by the process of claim 15.

17. The method of claim 15 wherein said source genetic material is human genetic material.

18. The method of claim 16 wherein said target cell is a CHO cell nucleus.

19. A genetic recombination method, comprising the steps of:
securing source genetic material,
cutting source genetic material,
acquiring cut source genetic material,
transporting cut source genetic material to a target location adjacent to target genetic material,
micro-beam welding said source genetic material to said target genetic material to create combined genetic material, and
micro-beam transfecting said combined genetic material into a target cell nucleus.

20. The combined genetic material product created by the process of claim 1.
